(19) United States
(12) Patent Application Publication
(10) Pub. No.: US 2014/0221500 A1
(43) Pub. Date: Aug. 7, 2014

(54) HIGHLY CONCENTRATED ASTAXANTHIN FOR TOPICAL APPLICATION

(71) Applicant: Wade Cheng, S. San Francisco, CA (US)

(72) Inventor: Wade Cheng, S. San Francisco, CA (US)

(21) Appl. No.: 13/757,317

(22) Filed: Feb. 1, 2013

(51) Int. Cl. A61K 8/35; A61Q 19/00

(52) U.S. Cl. CPC A61K 8/35; A61Q 19/00
USPC 514/691

(57) ABSTRACT

A method for obtaining and topically applying a customized resulting mixture, wherein the customized resulting mixture contains astaxanthin at an individualized concentration, the method comprising: placing in a mixing place a desired amount of a highly concentrated astaxanthin composition of known astaxanthin concentration, the astaxanthin concentration being between about 0.01% to about 5% by weight; placing a proportional amount of a substance near the highly concentrated astaxanthin composition; mixing the substance with the highly concentrated astaxanthin composition to obtain the customized resulting mixture; and, topically applying the customized resulting mixture.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of the Invention

The invention relates generally to personal care products, and more specifically, to highly concentrated astaxanthin compositions and methods of using them.

2. Description of the Related Art

Astaxanthin (CAS # 472-61-7; C₄₀H₅₂O₄; molecular weight 596.82) is a member of carotenoid and is a dark red fat-soluble nutrient. It possesses a long, double-bonded polyene chain which is a unique structure for superior anti-oxidation. It is believed to be the most powerful anti-oxidant in the world. Astaxanthin is rich in salmons and crustaceans (shrimps, lobsters, etc.) and some wild berries. The best natural source is microalgae called Haematococcus pluvialis. There are three stereoisomers of astaxanthin (3S,3′S; 3R,3′S and 3R,3′R). The natural astaxanthin (3S,3′S) is significantly more potent than other two stereoisomers (3R,3′S and 3R,3′R) due to biocompatibility. The synthetic astaxanthin contains all three stereoisomers.

Astaxanthin intake has shown many health benefits such as increasing strength and endurance, fighting against diabetes complication, reducing risk of cardiovascular disease, preventing cancers and assisting in neurodegenerative conditions such as Age-related macular degeneration (AMD), Alzheimer's and Parkinson's.

Astaxanthin has shown good anti-aging skin benefits such as antioxidation, cell protection, anti-inflammatory, internal sun protection and anti-pigmentation anti-wrinkles etc. The studies for these skin benefits were conducted by oral intake, or topical application with very low concentration (below 0.01% by weight or 100 ppm, limited by undesirable coloration) or combination of the both.

The concentration available for topical application of astaxanthin is very low (below 0.01%) due to undesirable coloration of the product appearance (red or orange color). Since topical application of astaxanthin does have excellent skin benefits, particularly at higher concentration, a new and improved astaxanthin composition and method of using it is needed in order to overcome the problems described above.

The problems and the associated solutions presented in this section could be or could have been pursued, but they are not necessarily approaches that have been previously conceived or pursued. Therefore, unless otherwise indicated, it should not be assumed that any of the approaches presented in this section qualify as prior art merely by virtue of their presence in this section of the application.

### BRIEF SUMMARY OF THE INVENTION

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In one exemplary embodiment a highly concentrated astaxanthin composition is provided. In another embodiment a method of using the highly concentrated astaxanthin composition is provided. The astaxanthin composition and the associated method provide a superior benefit to consumers by giving them a high degree of control over the mixing ratio of a resulting mixture containing the highly concentrated astaxanthin composition, and thus, over the undesirable skin coloration, while taking advantage of the great benefits astaxanthin has to offer.

The above embodiments and advantages, as well as other embodiments and advantages, will become apparent from the ensuing description and accompanying drawings.

### DETAILED DESCRIPTION OF THE PREFERRED EMBODIMENTS

What follows is a detailed description of the preferred embodiments of the invention in which the invention may be practiced. Reference will be made to the attached drawings, and the information included in the drawings is part of this detailed description. The specific preferred embodiments of the invention, which will be described herein, are presented for exemplification purposes, and not for limitation purposes. It should be understood that structural and/or logical modifications could be made by someone of ordinary skills in the art without departing from the scope of the invention. Therefore, the scope of the invention is defined by the accompanying claims and their equivalents.

Disclosed herein is a highly concentrated composition of Astaxanthin (preferably about 0.01% to about 5% by weight; more preferably about 0.05% to about 5% by weight), which can be miscible with water, gel, serum, lotion, cream, ointment or other cosmetic, skin, or personal care product, at any mixing ratio desired by a person, for topical application for skin care, anti-aging purpose or the like. The undesirable coloration of the Astaxanthin in the mixture can be minimized, or made less visible, by completing the process on, for example, a person's palm before topical application.

The highly concentrated composition of Astaxanthin (particularly natural Astaxanthin) may be an oil or water/oil mixture and may be used for topical use for skin care or personal care purposes as it will be explained in more details below. The concentrated astaxanthin mixture contains astaxanthin preferably between about 0.01% and about 5% by weight and more preferably between about 0.05% and about 5% by weight; thus, the concentration is much higher than that of personal care products containing Astaxanthin available on the market.

The highly concentrated Astaxanthin mixture is particularly suitable for mixing with any cosmetic solution, gel, serum, lotion, cream, ointment and the like, in any ratio desired by the user, before topical application. The mixing process is easily achieved by a person/user on his/her hands or palms, or in a small container, under ordinary conditions (i.e., no special conditions or tools are necessary). No elevated temperature, special agents or high-shear mixing or special agents are required to form a customized, individualized mixture or final product for topical application.

Because of the person/user's control over (1) where to mix the highly concentrated Astaxanthin composition with the personal care product, and (2) over the ratio between the concentrated Astaxanthin composition and the personal care product, the color of the resulting mixture may be much less visible on the person's skin after application, thus making the resulting mixture more desirable for personal care, than when using a product available on the market, containing Astaxanthin of a given, unchangeable concentration, from a cosmetic container such as a jar, tube, bottle or the like.

Thus, the product disclosed herein (the highly concentrated Astaxanthin composition) offers a high degree of flexibility to the person using the product. This is very important as skin absorption, reaction and/or coloration typically differs from person to person; thus, the choice is left with the person using the product to use a more or less concentrated resulting mixture, based on, for example, personal cosmetic/esthetic preferences. For example, a person with light skin who likes to have a darker looking skin may choose to use a more concentrated resulting Astaxanthin mixture, and vice versa.

Furthermore, not only the cosmetic objectives, such as the one described above, but also the health objectives may differ from person to person. For example, a person may want to ensure that more Astaxanthin is absorbed through her skin after a particular application, and that may require a resulting mixture of higher Astaxanthin concentration. Thus, the concentration of the resulting mixture needs to be customizable by the user to accommodate for these situations as well. Hence, for all the above reasons, it is important that a person is provided the option to customize/individualize the resulting mixture, which the disclosed product does.

In order to obtain the highly concentrated Astaxanthin composition disclosed herein, for example, commercially available raw material such as dry Astaxanthin powder (1-10% by weight), or a liquid of Astaxanthin in oleoresin (1-10% by weight), oleoresin being a naturally occurring mixture of an oil and a resin extracted from various plants, such as pine or balsam fir, may be dissolved or dispersed in vegetable oil (e.g., 5-10% by weight, based on the concentration of the raw material and of the final concentration of the Astaxanthin composition desired), such as sunflower seed oil, sesame oil, avocado oil, peanut oil or the like; then, the composition is preferably mixed with emulsifiers and/or surfactant (e.g., about 15-25% by weight) such as but not limited to sorbitan monooleate (Span 80), Polysorbate 20 (Polyoxyethylene (20) sorbitan monolaurate), Polysorbate 40 (Polyoxyethylene (20) sorbitan monopalmitate), Polysorbate 60 (Polyoxyethylene (20) sorbitan monostearate), Polysorbate 80 (Polyoxyethylene (20) sorbitan monooleate) or their mixture. Therefore, the obtained concentrated Astaxanthin in oil base become miscible with water (because of the surfactants), cosmetic solution, gel, serum and cream, ointment and the like, at any ratio desired by a person for topical application.

In addition, vitamin E (1-4% by weight) or tocopherol may be added into the oil base mixture to stabilize Astaxanthin and to achieve an enhanced synergistic effect.

A more preferable concentrated Astaxanthin composition is oil and water mixture and, if, for example, the raw material is Astaxanthin in oleoresin having a concentration of 10% by weight and the final concentration desired of the Astaxanthin composition is 1%, it may include about 15-25% by weight water, about 25-35% by weight oil and/or water miscible oil (hydrogenated oil), about 5-15% by weight propylene glycol, about 15-25% by weight surfactant(s) and/or emulsifier(s), and about 1-5% by weight vitamin E, and/or thickener. Such emulsion or oil/water mixture could be more miscible with cosmetic solution, gel, serum, cream, and the like, in comparison with the concentrated astaxanthin just in an oil base (without water), given that many such personal care products are water based. Therefore, it may be more suitable for topical application using personal care products. Furthermore, it should be apparent that the oil/water Astaxanthin composition is more miscible with water as well.

Thus, again, it should be observed that, the concentrated Astaxanthin composition disclosed herein is miscible to cosmetic serums, lotions, creams, and so on, for direct application by a person at desired, custom, individualized ratios, as opposed to simply adding the Astaxanthin at very low concentration into personal care products during the production.

It should be understood that the product disclosed herein (the highly concentrated Astaxanthin composition) may be sold separately, or, optionally, it may be packaged and sold as a set or kit including a container with the highly concentrated Astaxanthin composition, one or more personal care products to mix with, color charts depicting color coded concentration levels when mixing with a specific personal care product included in the set, measuring cups, mixing instructions, and so on.

It should be also understood that while the preferred concentrated Astaxanthin composition disclosed herein contains preferably between about 0.01% and about 5% by weight and more preferably between about 0.05% and about 5% by weight, higher (or possibly even lower) concentration may be used following the disclosed process, with similar results, thus, without departing from the scope of the invention.

What follows is an example of a typical process of using the product disclosed herein. These steps may be included in the user instructions accompanying the product.

### EXAMPLE 1

Step 1: Using a dropper, place a desired amount of the highly concentrated Astaxanthin composition in the palm or hand, or alternatively, a small container;

Step 2: Using a dropper or spatula, place a desired amount of personal care product (e.g., facial cream) near the highly concentrated Astaxanthin composition; optionally, a guide may be provided to be followed when the user performs Steps 1 and 2 (e.g., use one dropper of the highly concentrated Astaxanthin composition and 1-5 dropper or spatula of the personal care product to decrease the concentration of the Astaxanthin in the resulting mixture down to 50%-15%)

Step 3: Mix the two substances to obtain a resulting mixture by rubbing or stirring with spatula and,

Step 4: Apply the resulting mixture on skin (e.g., facial skin)

It may be advantageous to set forth definitions of certain words and phrases used in this patent document. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

As used in this application, “plurality” means two or more. A “set” of items may include one or more of such items. Whether in the written description or the claims, the terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases with respect to claims. Use of ordinal terms such as “first,” “second,” “third,” etc., in the claims to modify a claim element does not by itself connote any priority, precedence or order of one claim element over another or the temporal order in which acts of a method are performed. These terms are used merely as labels to distinguish one claim element having a certain name from another element having a same name (but for use of the ordinal term) to distinguish the claim elements. As used in this application, “and/or” means that the listed items are alternatives, but the alternatives also include any combination of the listed items.

Throughout this description, the embodiments and examples shown should be considered as exemplars, rather than limitations on the composition, product and processes disclosed or claimed. Although the examples involve specific combinations of method acts or product elements, it should be understood that those acts and those elements may be combined in other ways to accomplish the same objectives.

For means-plus-function limitations, if any, recited in the claims, the means are not intended to be limited to the means disclosed in this application for performing the recited function, but are intended to cover in scope any means, known now or later developed, for performing the recited function.

Although specific embodiments have been illustrated and described herein for the purpose of disclosing the preferred embodiments, someone of ordinary skills in the art will easily detect alternate embodiments and/or equivalent variations, which may be capable of achieving the same results, and which may be substituted for the specific embodiments illustrated and described herein without departing from the scope of the invention. Therefore, the scope of this application is intended to cover alternate embodiments and/or equivalent variations of the specific embodiments illustrated and/or described herein. Hence, the scope of the invention is defined by the accompanying claims and their equivalents. Furthermore, each and every claim is incorporated as further disclosure into the specification and the claims are embodiment(s) of the invention.

What is claimed is:

1. A method for obtaining and topically applying a customized resulting mixture, wherein the customized resulting mixture contains astaxanthin at an individualized concentration, the method comprising: placing in a mixing place a desired amount of a highly concentrated astaxanthin composition of known astaxanthin concentration, the astaxanthin concentration being between about 0.01% to about 5% by weight; placing a proportional amount of a substance near the highly concentrated astaxanthin composition; mixing the substance with the highly concentrated astaxanthin composition to obtain the customized resulting mixture; and, topically applying the customized resulting mixture.

2. The method of claim 1, wherein the astaxanthin concentration is between about 0.05% to about 5% by weight.

3. The method of claim 1, wherein the highly concentrated astaxanthin composition comprises oil, water, and at least one member of a group consisting of a surfactant and an emulsifier.

4. The method of claim 1, wherein the highly concentrated astaxanthin composition comprises oleoresin, oil and at least one member of a group consisting of a surfactant and an emulsifier.

5. The method of claim 1, wherein the mixing place is the palm of a person practicing the method.

6. The method of claim 1, wherein the mixing place is a container, and wherein the method is practiced by a person other than the person being the subject of the topical application of the customized resulting mixture.

7. The method of claim 1, wherein the proportional amount of the substance is about twice the amount of the desired amount of the highly concentrated astaxanthin composition, such that the astaxanthin concentration in the customized resulting mixture is about one third of the known astaxanthin concentration in the highly concentrated astaxanthin composition.

8. The method of claim 1, wherein said substance is water.

9. The method of claim 1, wherein said substance is skin lotion.

10. A highly concentrated astaxanthin composition wherein the astaxanthin concentration is between about 0.01% to about 5% by weight, wherein the astaxanthin is dispersed in a mixture of oil and water, and wherein the composition comprises at least one member of a group consisting of a surfactant and an emulsifier.

11. The highly concentrated astaxanthin composition from claim 10, wherein the astaxanthin concentration is between about 0.05% to about 5% by weight.

12. The highly concentrated astaxanthin composition from claim 11 wherein the water concentration is between about 15% to about 25% by weight, the oil and water miscible oil concentration is between about 25% to about 35% by weight and the at least one member's concentration is between about 15% to about 25% by weight.

13. A highly concentrated astaxanthin composition wherein the astaxanthin concentration is between about 0.01% to about 5% by weight, and wherein the composition comprises oleoresin, oil, and at least one member of a group consisting of vitamin E, a surfactant and an emulsifier.
